(19) United States
(12) Patent Application Publication
(10) Pub. No.: US 2005/0112115 A1
(43) Pub. Date: May 26, 2005

(54) Surface roughness quantification of pharmaceuticals, herbal, nutritional dosage forms and cosmetic preparations

(76) Inventor: Mansoor A. Khan, Amarillo, TX (US)

Correspondence Address: Jennifer P Yancy;Jones Tullar & Cooper, P O Box 2266, Eads Station, Arlington, VA, 22202, US

(21) Appl. No.: 10/480,687

(22) PCT Filed: May 29, 2002
(86) PCT No.: PCT/US02/16685

(30) Foreign Application Priority Data
May 29, 2001 (US) 60293525

(51) Int. Cl. A61K 39/395; A61K 9/52

(52) U.S. Cl. 424/130.1; 424/457

(57) ABSTRACT

Dosage forms are identified based on a comparison of surface roughness parameters. One or more of the following surface roughness parameters are measured: 1) mean peak to valley height (Rz); 2) geometric average height from a mean line (Rq); 3) maximum profile peak height (Rp); 4) roughness depth (Rt); 5) and arithmetic mean roughness (Ra). The surface roughness parameters of a first dosage form are determined and compared to the surface roughness parameters of a second dosage form. This method provides a way of “fingerprinting” dosage forms to help identify adulterated and misbranded drugs. In addition, a variety of characteristics relating to composition and process for making the dosage form may be determined by the quantitative method.

### CROSS REFERENCE TO RELATED APPLICATION

This application claims the benefit, under 35 U.S.C. 119(e), of U.S. Provisional Application No. 60/293,525 filed May 29, 2001, the contents of which are incorporated herein by reference.

### BACKGROUND OF THE INVENTION

1. Field of the Invention

Surface roughness parameters of a dosage form are quantitatively measured in order to determine one or more characteristics of the dosage form.

2. Background Information

Several classes of natural, synthetic and biotechnologically-derived drugs are being discovered by scientists all over the world. In order for these drugs to become effective, they need to be available in the body between a certain minimal effective concentration and a particular toxic concentration. With certain drugs, the rate at which the drug substance becomes available can be very high or none at all. For example, a bioavailability difference of 10 to 100% has been shown in commercial digoxin preparations. In most of the cases, the variability in bioavailability is directly related to formulation considerations. An important role of product development is to make safe and effective dosage forms by a careful selection of additives, manufacturing techniques, and by a thorough consideration of the variables affecting the composition, stability and utility of the product.

Although there are a variety of dosage forms available, the most common dosage form is a tablet. Traditionally, tablets are prepared either by direct compression of a drug with additives or by a wet granulation process. The wet granulation process involves the mixing of all ingredients, granulation by using a binder, drying, dry granulation, lubrication and compression. Depending upon the end requirements, tablets are coated by film coating, chocolate coating, or sugar coating. The common problems associated with tabletting are capping and lamination, picking and sticking, mottling, weight variation, punch variation, hardness variation, friability, and variations in disintegration and dissolution. Film defects reported are sticking and picking, roughness, orange peel effect, bridging and filling, blistering, color variation and cracking.

To design tablets and later monitor tablet production quality, quantitative evaluation and assessment of a tablet's chemical, physical and bioavailability properties must be done. Currently

tablets are evaluated by their general appearance, size and shape, organoleptic properties, hardness, friability, weight variation, disintegration, dissolution and content uniformity.

Prior techniques for assessing the picking, sticking or roughness in the surface measurements include a qualitative estimation of the surface of the tablets. This evaluation procedure can be very subjective; therefore, a quantitative roughness measuring method should be of considerable interest to pharmacy students, pharmaceutical companies and also to the State and Federal Regulating Agencies. Besides measuring the extent of picking and sticking quantitatively, the roughness measurements would aid in the determination of optimum level of coating solution or dispersion application on tablets, evaluation of the effect of moisture and other variables on the quality of surface smoothness of coated and uncoated tablets, and for the forensic purpose wherein the adulterated tablets can be easily identified.

The roughness of uncoated tablets can be caused by crystalline behavior of ingredients, retention of undesired levels of moisture, surface drying, or uneven compression pressures. For coated tablets, roughness can be due to blistering of the film, orange peeling, uneven application of coating solutions or dispersion, and mottling. Consequently, a need exists for a method of quantitatively determining the surface roughness characteristics of a dosage form to permit evaluation of various properties of the dosage form, including methods of production, physical properties and chemical compositions.

### BRIEF SUMMARY OF THE INVENTION

Various characteristics of dosage forms may be evaluated by comparing one or more surface roughness parameters of at least a first dosage form to corresponding surface roughness parameters of at least a second dosage form. The surface roughness parameters used for this purpose are one or more of the following: 1) mean peak to valley height (Rz); 2) geometric average height from a mean line (Rq); 3) maximum profile peak height (Rp); 4) roughness depth (Rt); 5) and arithmetic mean roughness (Ra). Once the surface roughness parameters are obtained, a surface roughness comparison using the measured values is determined to provide a roughness differential, and this roughness differential is then used to determine a specified characteristic of the dosage form. These surface roughness parameters are preferably determined by measuring peaks generated by a perthometer. Quantitative surface roughness measurements using a combination of several surface roughness parameters have an important application in the area of dosage form design and evaluation, for when the type and concentration of ingredients, and the processes for making the dosage form are changed, the roughness profiles of the dosage form will also change. This change in profile can be used to identify the ingredients and processes used in manufacturing the dosage form.

The measured surface roughness parameters are used to identify a variety of characteristics of a dosage form including, but are not limited to, the following: 1) the ingredients and grades of material used to make the dosage; 2) the optimum amount of coating solution or dispersion needed for a dosage form; and 3) the processes used to make the dosage form. In essence, these surface roughness parameters provide a “fingerprint” of a dosage form which specifically identifies that dosage form. In order to obtain the most information about a dosage form, and thus its “fingerprint”, all five surface roughness parameters are measured; however, valuable information about certain characteristics of a dosage form may be obtained by using less then all five surface roughness parameters. Specific identification of a dosage form may be used to monitor the production quality of the dosage form, including the chemical and physical properties of the dosage form and bioavailability of the drug in the dosage form. In addition, the “fingerprint” of the dosage form helps determine whether an unauthorized production of the dosage form has been performed or whether a dosage form has been misbranded. A surface roughness comparison may be used to compare surface roughness parameters of a known dosage form to an unknown dosage form to determine, for example, whether a drug is being properly manufactured, whether the drug has been properly marked, or whether the drug is a counterfeit. In addition, the surface roughness parameters of at least two unknown dosage forms may be compared in order to determine, for example, the optimum amount of coating solution for a dosage form, the effects of direct compression on a dosage form, the effects of wet granulation on a dosage form, and the rate of dissolution of a drug in the dosage form.

The quantitative method of the present invention for comparing the surface roughness parameters of dosage forms thus has a variety of applications. The surface roughness parameters may be used as a method to “fingerprint” coated and uncoated pharmaceutical, herbal and nutritional dosage forms as well as cosmetic preparations. The nature and concentration of the ingredients used to formulate the dosage form may be identified by comparing the surface roughness parameters of known dosage forms with sample dosage forms obtained from the production line. Additionally, the surface roughness parameters of a known dosage form may be used to ensure the quality of a process used for preparing dosage forms. Using surface roughness parameters is useful in determining the coating end points of both organic and aqueous based coatings of dosage forms. Surface roughness parameters may also help determine the exact grade of any ingredients in the formulation of the dosage form, coating defects of the dosage form, compression defect in the dosage form, lubricant mixing times needed to obtain a uniform dosage form, and the order and time of mixing of ingredients in gels, pastes, creams, ointments, plasters and cataplasms.

By using a quantitative method for determining the surface roughness of dosage forms, more precise characteristics may be determined as compared to prior subjective determinations which evaluate the surface roughness of the dosage form using qualitative and visual testing techniques. Surface roughness techniques which have been traditionally used in the testing of tools and automobile parts can now be applied to surface testing of dosage forms. Quantitative testing solves the “forensic” problem of differentiating an original dosage form from legal or illegal duplicates and adulterated or misbranded products. For a given dosage form, the present method will help determine the ingredients and process employed for preparing the dosage form without actually having to damage the dosage form.

### BRIEF DESCRIPTION OF DRAWINGS

The features and advantages of the present invention will become apparent from the following detailed description of a preferred embodiment thereof, taken in conjunction with the accompanying drawings, in which:

FIG. 1. illustrates how the surface roughness parameters are determined based on an Abbott-Firestone curve;

FIG. 2 is a graph showing the effect of Avicel coating thickness on surface roughness;

FIG. 3 shows a representative surface topography, P, W, and R profiles of self-nanoemulsified tablets, obtained by a Mahr perthometer concept surface-measuring instrument; and

FIG. 4 is a graph of surface roughness parameters as a function of CAB pseudolatex coating weight gain.

### DETAILED DESCRIPTION OF PREFERRED EMBODIMENTS

In accordance with the present invention, a method for evaluating the characteristics of dosage forms includes comparing the surface roughness parameters of at least a first dosage form to the corresponding surface roughness parameters of at least a second dosage form. The surface roughness parameters are selected from the following surface roughness parameters: mean peak to valley height (Rz), geometric average height from a mean line (Rq), maximum profile peak height (Rp), roughness depth (Rt) and arithmetic mean roughness (Ra), and a characteristic of a dosage form is determined by using one or more of these surface roughness parameters. However, to obtain a more complete “fingerprint” of the dosage form a measurement of all five surface roughness parameters is preferred. Then foregoing parameters are preferably determined by measuring peaks generated by a perthometer.

A characteristic of a dosage form is evaluated by determining at least one surface roughness parameter of a first dosage form, determining at least one surface roughness parameter of a second dosage form, comparing surface roughness parameters of the first dosage form to corresponding surface roughness parameters of the second dosage form to obtain a roughness differential, and evaluating the characteristic of the second dosage form based on the roughness differential. The surface roughness parameters for the first dosage form and the second dosage form are selected from the group including Rz, Rq, Rp, Rt, and Ra. The more surface roughness parameters that are determined and compared, the more information relating to the characteristic of the dosage form is obtained. Although it is preferred to use all five surface roughness parameters in order to obtain the most complete information about the dosage form, one or more surface roughness parameters may also be used to evaluate various characteristics of a dosage form. The surface roughness parameters of the first dosage form may be known surface roughness measurements that have been previously determined in order to provide a standard. The standard may then be compared with measured surface roughness parameters of the second dosage form in order to evaluate various characteristics of the second dosage form. This method for determining the characteristic of a dosage form may be used to determine characteristics of a coating of a dosage form, characteristics of an ingredient in a dosage form, determining a process for preparing a dosage form, determining a release characteristic of a drug in a dosage form, and a multitude of other applications. Although many of the following illustrations and examples compare the surface roughness parameters of tablets, other dosage forms including, but not limited to, cataplasms, coated beads, uncoated beads, granules, paste, creams and ointments may be evaluated using the present invention.

Surface roughness parameters may be used to evaluate a variety of characteristics of a dosage form. However, the following illustrations will focus on the measurement of surface roughness parameters to evaluate the effect of diluents, binders, binder concentrations, glidant type and concentration, lubricant type and concentration, compression pressure and machine speed on the picking and sticking, and release profiles of a therapeutic agents for a particular dosage form. Furthermore, although the surface roughness parameters of a variety of dosage forms, containing a variety of drugs and other additives, may be measured and compared in accordance with the invention, for illustrative purposes, only certain model drugs such as indomethacin, prazocin, and potassium chloride, using additives such as lactose, calcium sulfate, dicalcium phosphate, starch, gelatin, talc, and magnesium stereate will described herein.

The following tests provide illustrations of how certain characteristics of dosage forms are evaluated. In accordance with the invention, a series of these tests are performed on a plurality of dosage forms in order to identify a given characteristic of the dosage forms. Measurements of the surface roughness of the plurality of dosage forms are then evaluated to compare these measurements with the corresponding characteristic of the dosage form so that the effects of characteristics such as direct compression, wet granulation, concentration of ingredients, and osmotically-controlled drug release on surface roughness are obtained. In addition, the following illustrations show how measurements of surface roughness parameters may be used to optimize the amount of coating/dispersion of a dosage form and evaluate the drug:excipients interaction of the dosage form. The tests are described as follows:

Direct Compression Effect on a Dosage Form

In order to test the direct compression effect of a dosage form, appropriate amounts of drugs and diluents, such as dicalcium phosphate, are mixed thoroughly in a V-blender. Magnesium stereate and talc are mixed in different ratios and the dosage form, in this instance tablets, is compressed at different compression pressures. The effects of compression pressures, diluents, lubricants, and glidant concentration on the surface roughness and the dissolution are evaluated.

Wet Granulation Effect on a Dosage Form

Next, the effect of wet granulation is evaluated by mixing appropriate amounts of drugs with diluents and binder solutions. The wet mass is preferably passed through a #12 mesh and dried in an oven. The dried granules are preferably passed through a #16 mesh and after adding magnesium stereate and talc, are mixed in the V-blender. The mixture thus obtained is compressed in a Stokes' Rotapress® (an automatic 16-station Stoke's Rotary Tablet Press from the Wyeth-Ayerst Company at Pearl River, N.Y.). The effects of the type of binder used, binder concentration, compression pressure, additives, lubricant and glidant ratios, type of lubricants, mixing times, and moisture levels on the surface roughness, hardness and dissolution of tablets are evaluated. The release kinetics are fit into appropriate models to evaluate the mechanism of release at different roughness indices. An Ohaus Moisture Balance is used to evaluate the moisture levels prior to compression.

Release Characteristic of an Osmotically-Controlled Release Tablet

Bilayered tablets comprising a layer of osmotic agents, for example sodium chloride with a polymer, and a second layer of drugs with another polymer are compressed in a Carver-Press, and their surface roughness and hardness are evaluated. The compressed tablets along with placebo tablets (the latter are compressed by Stoke's Rotapress) are coated with a semipermeable membrane by using a Strea I fluid-bed coater. Once coated, apertures of different sizes and shape are made in the drug layer so that, upon contact with the dissolution media or gastrointestinal fluids, the osmotic agent in the lower layer imbibes water, swells the tablets and releases the drug through the aperture depending upon the osmotic pressure developed inside. The effect of surface area and surface roughness on the tablet hardness and dissolution, and the effect of size and shape of the aperture on coating efficiency and dissolution are evaluated.

Dissolution Effects of a Drug in a Dosage Form

All the tablets are subjected to dissolution evaluations as specified in the monograph of model drugs. As an example, dissolution of indomethacin tablets is performed in a USP rotating basket apparatus with a spindle speed of 100 rpm. The dissolution medium employed is 900 mL of simulated intestinal fluid at 37° C. At appropriate time intervals, samples are allowed to flow through the spectrophotometer by an automated assembly to monitor the amount of drug dissolved. The preferred automatic dissolution equipment is a diode array spectrophotometer from Novartis Pharmaceuticals Corporation, New Jersey. Cumulative percent of drug dissolved is plotted against the time. By suitable mathematical models, release profiles are examined, and appropriate modeling is performed.

Measuring Surface Roughness Parameters

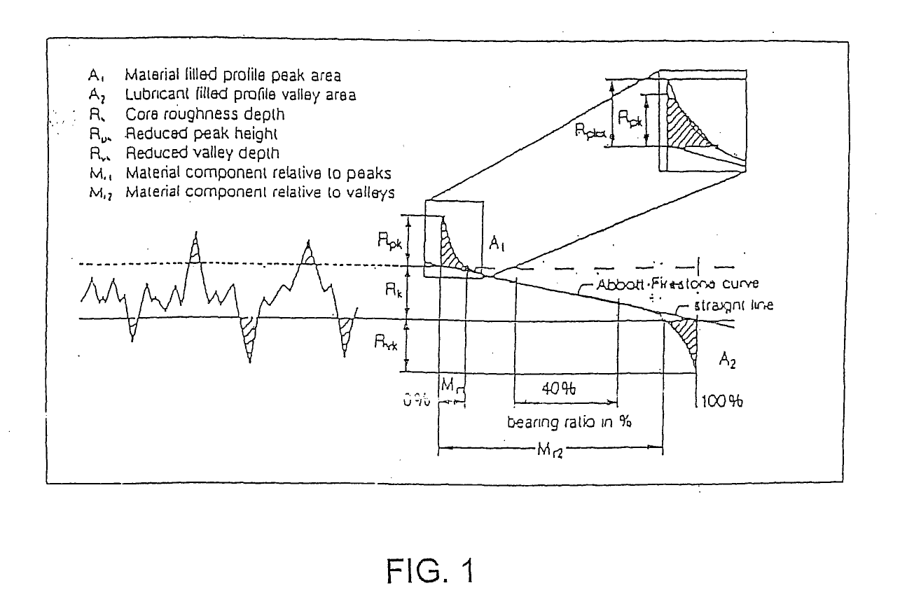

After calibrating a Perthometer S8P, tablets are mounted on the worktable to measure the surface roughness parameters. Proper stylus type and measuring areas are selected after preliminary investigations and kept constant for all measurements. After tracing suitable areas, surface roughness parameters are obtained, evaluated and compared. The Perthometer S8P acquires, graphs, presents and evaluates the surface profiles according to established international test specification (DIN 4776). The surface roughness parameters are based on Amplitude Density Function (ADF) and the Abbott-Firestone Curves (TPK). The ADF measurements are standardized (DIN 4762/IE), and the TPK parameters are obtained from an Abbott-Firestone Curve, as shown in FIG. 1.

The surface roughness parameters evaluated are mean peak to valley height (Rz), geometric average height from a mean line (Rq), maximum profile peak height (Rp), roughness depth (Rt) and arithmetic mean roughness (Ra). In the case of osmotically-controlled delivery systems, three-dimensional pictures of the aperture and surrounding areas are also obtained.

Tablets may also be evaluated for their hardness on an Erweka hardness tester, friability on an Erweka friabilitor, and disintegration on an Erweka disintegration apparatus.

Determining the Optimum Amount of Coating Solution and Dispersion for Coated Dosage Forms

Surface roughness parameters are also used to evaluate the optimum amounts of coating solution or dispersion in film coated dosage forms (i.e. tablets). Tablets are prepared by the wet granulation process as outlined above and coated with coating solutions prepared with ethyl cellulose in a suitable solvent. Appropriate amounts of plasticizer and other additives, if needed, are added. Then coating operations are performed in a Strea-I fluid-bed coater. After the addition of every layer of the coating, a few tablets are removed, dried at room temperature, and evaluated to determine their surface roughness parameters by using a perthometer. If the tablets are smooth initially, improper or insufficient coating will produce higher surface roughness values. Once the coating is complete, the surface roughness values should decrease. Commercially available coating dispersions of Eudragit® are also applied to a separate batch of tablets. The exact amount of dispersion needed and the film problems are studied by using the perthometer.

The Effect of Drug:Excipients Interactions

Quantitative surface roughness parameters are used to study the effect of drug:excipients interactions on the surface and release profiles of dosage forms. Interaction of the drugs and excipients are evaluated with the help of a differential scanning calorimeter (DSC) from Knoll Pharmaceuticals Corporation, Shreveport, La. Before operation, the DSC is calibrated with approximately 3 to 7 mg of indium standard. The drug substances are accurately weighed in small aluminum pans. The pans are covered with aluminum lids and sealed. An empty aluminum pan similarly sealed is used as a reference. Samples are heated from 50 to 200° at a scan rate of 10° C. in an atmosphere of nitrogen. After completion of the run, thermograms are normalized to one milligram weight. Peak onset (melting point), and heat of fusion (ΔH_{f}) are measured. Thermograms of all additives are obtained separately. The 50% mixtures of drugs and additives are obtained separately. From the differences of endothermic peaks of the drug, additives and their mixtures, interactions are determined. As an example, previous studies have indicated an interaction of magnesium stearate with ibuprofen. It is known that magnesium stearate is the most widely used lubricant, but, it is not known if the interaction is of any serious concern. Therefore, a measurement of the surface roughness parameters of the tablets should provide an idea of the behavior of the tablets with regard to their surfaces and dissolution with time. After determining the interactions, tablets are prepared by the direct compression method with the same additives, and surface roughness and dissolution studies are performed as outlined previously.

The following examples illustrate specific characteristics of a dosage form that may be determined by measuring the surface roughness parameters of the dosage form. Although a series of evaluations may be preferred in order to determine a number of these characteristics, surface roughness parameters may be measured in order to determine a specific characteristic of the dosage, including the relationship between coating weight gain and roughness and the effects of formulation on the surface roughness, as illustrated in the following examples.

### EXAMPLE 1

The following Example illustrates the relationship between coating weight grain and roughness parameters of Microcrystalline Cellulose Avicel®. The testing shows the various properties of Avicel® as they relate to roughness parameters. The testing evaluates the change in surface roughness of the dosage form as a function of wet granulation, direct compression and compression pressure.

The perthometer used was a state-of-the-art Mahr perthometer, a complete package of motor driven contact stylus, X/Y-table PZK for mounting the tablets, and software for processing the data. The contact stylus scanned over an area of 3.0 mm² with a tracing length of 1.75 mm to produce 201 profiles. All the surface roughness parameters were calculated for every profile, and the mean and average of all 201 profiles were collected to represent the complete topography.

Bilayered tablets were prepared and a custom-designed cellulose acetate pseudolatex dispersion was applied for osmotic controlled drug delivery. Tablets were taken out in periodic intervals of coating weight gain and the roughness was characterized.

Different types of Avicel® were compressed at 0.5 ton and one and two percent of magnesium stearate and talc were added as glidant and lubricant.

Avicele granules were prepared and compressed at 0.5 ton pressure and the surface roughness was compared with that of direct compression.

Directly compressible material Avicel® 101 and Avicel® 102 were compressed at 0.5, 1.25, and 2.0 ton pressure (carver semiautomatic press) and the surface roughness parameters were measured. One and two percent of magnesium stearate and talc were added as glidant and lubricant.

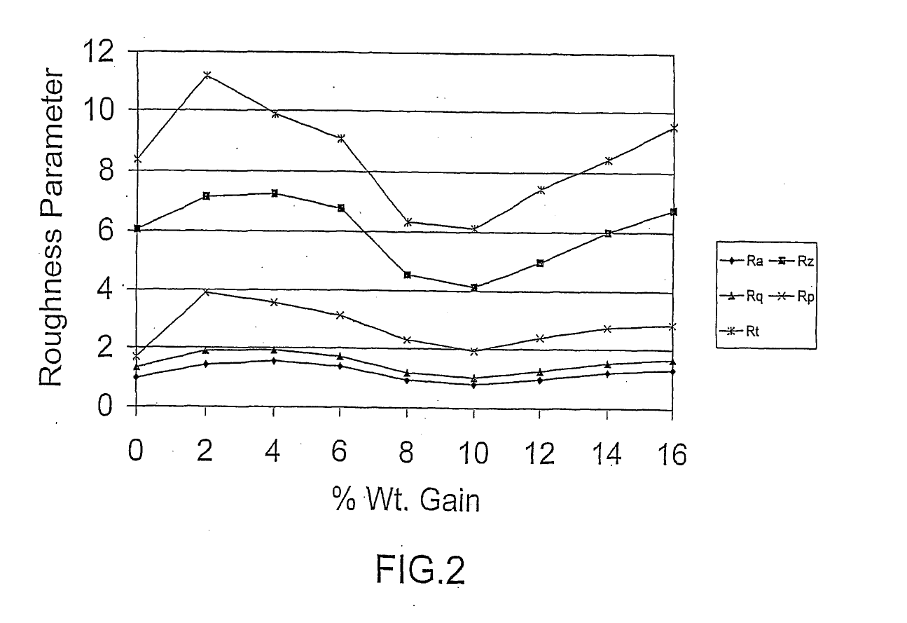

As shown in FIG. 2, the surface roughness of the membrane increased with the increase in coating weight gain until 2 to 4% wt. gain and then started decreasing. The smoothest surface was found at 10% wt. Gain (see FIG. 2). Any further increase in coating weight gain gradually increased the surface roughness. The Figure clearly demonstrates that a uniform membrane was achieved at 10% wt. gain and that any further increase in coating thickness would decrease the uniformity, resulting in an unpredictable release pattern. For predictable membrane-controlled dosage formulations, not only the weight gain of the membrane matters but also the uniformity of the membrane.

Avicel® PH-101 has smaller mean particle size than Avicel® PH-102, resulting in higher volumetric flow. Avicel® PH-301 and 302 are similar to Avicel PH-101 and 102, except for 33% more bulk density, thus helping to avoid powder stratification and segregation. Avicel® PH 105 is the finest powder available in the Avicel family whereas Avicel® PH 200 has the largest particle size. Thus, these grades of Avicel® have distinctive properties such as nature and quantity of the active drug, desirability of the product as well as the process variables, that allow the formulator to select the appropriate grade according to the application.

Among all the tablets compressed out of Avicel®, PH-105 registered the lowest roughness, which may be because it has the smallest particle size. As shown in Table 1, tablets compressed out of PH-101 had maximum roughness. This may be due to the needle shaped particles of Avicel PH-101. This property of PH-101 also reflects a mass flow (0.56 Kg/min) that is much lower than any other Avicel® used. Tablets made with PH-105 have the lowest surface roughness properties due to the fine particle size (20 μm) and relatively regular particle shape. Though the particle size of Avicel® PH-200 is 3.6 times greater than PH-101, the tablets made with PH-200 were much smoother than the PH-101, which was primarily due to the granule shaped particles of PH-200.

TABLE 1
Effect of Different Grades of Avicel ® on Roughness

| Parameter | 101 | 102 | 301 | 302 | 105 | 200 |
|---|---|---|---|---|---|---|
| Ra | 0.9 | 0.8 | 0.5 | 0.4 | 0.4 | 0.5 |
| Rz | 3.9 | 3.5 | 2.3 | 2.0 | 1.8 | 2.3 |
| Rq | 1.1 | 1.0 | 0.7 | 0.6 | 0.5 | 0.6 |
| Rp | 1.7 | 1.6 | 1.0 | 0.9 | 0.8 | 1.0 |
| Rt | 5.8 | 5.5 | 3.5 | 3.2 | 2.6 | 3.5 |

As shown in Table 2, tablets prepared by wet granulation (W) were much smoother than those prepared by direct compression (D). This may be due to the presence of water that makes the compression effective. The decrease in roughness was reflected on both Avicel® PH-101 and 102, suggesting independency of particle size.

The increase in compression pressure from 0.5 Ton to 1.25 Ton dramatically decreased the surface roughness (see Table 2). Nevertheless, a further increase in compression pressure did not decrease the roughness proportionately, perhaps because a decrease in roughness goes in parallel with an increase in the hardness of the tablet. There is a saturation point in the decrease in roughness, above which any additional compression pressure may not facilitate a reduction in smoothness.

TABLE 2
Effect of wet granulation and direct compression
on roughness parameters

| | PH-101 | | PH-102 | | PH-101-effect of CP | | |
|---|---|---|---|---|---|---|---|
| | D | W | D | W | 0.5 Ton | 1.25 Ton | 2 Ton |
| Ra | 0.9 | 0.6 | 0.8 | 0.6 | 0.9 | 0.3 | 0.3 |
| Rz | 3.9 | 2.8 | 3.5 | 2.7 | 3.9 | 1.4 | 1.2 |
| Rq | 1.1 | 0.8 | 1.0 | 0.8 | 1.1 | 0.4 | 0.3 |
| Rp | 1.7 | 1.2 | 1.6 | 1.1 | 1.7 | 0.6 | 0.5 |
| Rt | 5.8 | 4.6 | 5.5 | 4.7 | 5.8 | 2.0 | 1.8 |

The uniformity of the coating in membrane-controlled drug delivery systems such as osmotically controlled tablets can be very well identified by measuring the roughness parameters. The nature and type of the excipient can be identified by carefully creating a library of surface roughness parameters of several different excipients. Tablets made by wet granulation technique were much smoother than those prepared by direct compression. The roughness decreased with increases in compression pressure up to a certain point, and after that no proportionate decrease in roughness was observed with increase in compression pressure.

### EXAMPLE 2

Ubiquinone, also known as Coenzyme Q₁₀, is an important component of the mitochondrial respiratory chain. Because of the poor aqueous solubility, Coenzyme Q₁₀ (CoQ₁₀) presents a challenge when developing a formulation for oral administration. Many approaches have been used to improve the in vitro dissolution of CoQ₁₀, including complexation, preparation of redispersible dry emulsion, solid dispersion, and eutectic-based self-nanoemulsified drug delivery system (SNEDDS). A wax-like paste is formed when a eutectic-based SNEDDS of CoQ₁₀ is mixed with small quantities of the copolyvidone Kollidon VA 64. The effects of an adsorbed oily formulation on the surface roughness of the tablets can be determined.

A solid-state SNEDDS of CoQ₁₀ was prepared as follows: CoQ₁₀ and lemon oil at a ratio of 1:1 were accurately weighed into screw-capped glass vials and melted in a water bath at 37° C. Cremophor EL and Capmul MCM-C8 were added to the oily mix, each at a final concentration of 26.9% w/w. The resultant emulsion was mixed with a stirring bar until a transparent solution SNEDDS was obtained. The SNEDDS solution was allowed to cool to ambient temperature for 24 hours, until a viscous paste was obtained. Nanoemulsion-absorbed granular material was obtained from a mixture of SNEDDS paste, Kollidon VA 64, Glucidex IT 12, and Avicel at a ratio of 0.11:0.13:0.56:0.2, respectively. SNEDDS was mixed initially with Kollidon VA 64 using a mortar and pestle until a semisolid waxy paste was obtained. The mixture then was ground with Glucidex IT 12 in the mortar for 1 min to obtain the dry microemulsion-based granules. Finally, Avicel MCC was added to the granules and blended in a V-blender (Patterson-Kelley Co., E. Strousburg, Pa.) for 5 min. Six formulations were made, each with a different grade of Avicel MCC.

Microemulsion-adsorbed compacts were prepared using concave elongated punches (Natoli Engineering Co., St. Charles, Mo.). Tablets were made by compressing 1245 mg of powder, which corresponds to 30 mg in weight of CoQ₁₀, between the faces of the punch. Punches were mounted between the platens of a Carver press model C (Carver Inc., Wabash, Ind.) attached to a semiautomatic compression assembly model 2826 (Carver). The compaction pressure ranged from 15.6 to 312.3 Mpa. The dimensions of the compact were measured to ±0.01 mm using a dial thickness gauge (Lux Sci. Inst. Corp., New York, N.Y.). Punches were 0.750 in. long and 0.375 in. wide and provided tablets with an area of the curved segment equivalent to 0.0083 in.³ and a height of the curved surface above the central thickness equivalent to 0.06 in.

The roughness profiles for the upper and lower surfaces of the compacts were measured with a Mahr perthometer concept 6.3 surface texture-measuring instrument (Mahr Federal Inc., Cincinnati, Ohio). Tablets were mounted on the X/Y table and scanned with a contact PZK drive unit using the stylus method to move the tracing arm (model MFW-250) across the surface. A tracing length of 3.5 mm was used to obtain 51 profiles with a spacing of 112 μm. P-profile, waviness, and surface roughness parameters were computed for every profile, and the mean of all 51 profiles was collected. The following parameters were measured:
Ps (profile parameter): the mean distance between local peaks of the P-profile
Ws (waviness parameter): the mean distance between local peaks of the W-profile
Ra (roughness average): the arithmetic average of the roughness profile ordinates
Rz (mean roughness depth of the R-profile): the arithmetic average of roughness depths (i.e., the vertical distance between the highest peak and the deepest valley of consecutive sampling heights)
P-profile (primary profile): the mean line generated from the traced profile.

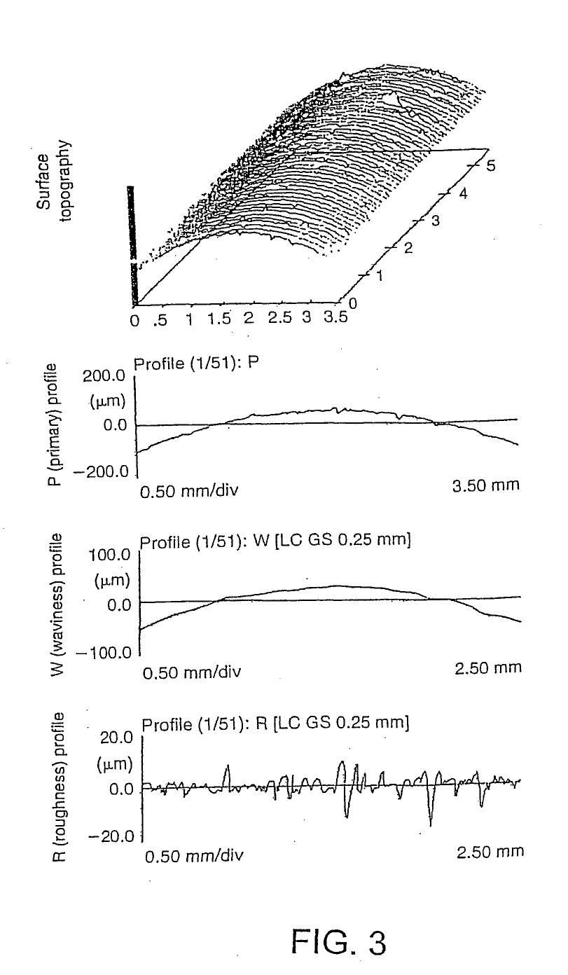

Using profile filters, P-profiles separate into long-wave (W-profile) and short-wave (R-profile) components. A representative surface topography, P, W, and R profiles obtained by the profilometer shown in FIG. 3.

The profile parameters measured for the compacts are listed in Table 3. Waviness of the lower surface of the tablets, exposed to the lower punch during compaction, was greater than that of the upper surface of the compacts. This is apparent from the W and P profiles given as Ws and Ps parameters, respectively. Higher values of the Ws parameter for the lower surface of the tablets might be the result of the segregation of the larger granules to the bottom of the die during powder filling. These granules are the Kollidon VA 64-based paste ground with maltodextrin. Segregation was visually evident by the higher degree of mottling of the lower surface caused by the colored granules when compared with the extragranular white Avicel MCC powder. However, no change in surface waviness was observed as a function of the initial Avicel MCC particle size.

TABLE 3
P, W, and R surface roughness parameters of the upper and lower surfaces of the
tablets as a function of the Avicel MCC grade added to the formulation.

| Avicel MCC | P-profile | | W-profile | | R-profile | | | |
|---|---|---|---|---|---|---|---|---|
| Grade Added to | Pₛ (μm) | | Wₛ (μm) | | Rₐ (μm) | | R_{z} (μm) | |
| the Formulation | Upper | Lower | Upper | Lower | Upper | Lower | Upper | Lower |
| Avicel PH-105 | 118.2 | 151.68 | 391.02 | 446.9 | 2.97 | 1.83 | 20.07 | 12.84 |
| Avicel PH-101 | 123.82 | 157.09 | 373.9 | 486.62 | 1.84 | 1.63 | 12.92 | 11.6 |
| Avicel PH-113 | 143.6 | 144.24 | 384.26 | 427.14 | 1.96 | 1.84 | 14.26 | 13.08 |
| Avicel PH-102 | 156.78 | 173.51 | 386.88 | 469.76 | 1.48 | 1.31 | 10.62 | 9.26 |
| Avicel PH-112 | 152.31 | 160.58 | 357.12 | 405.87 | 1.85 | 1.59 | 13.53 | 10.91 |
| Avicel PH-200 | 162.34 | 175.16 | 387.62 | 431.95 | 1.77 | 1.52 | 12.57 | 11.04 |

On the other hand, the P-profile measures both roughness and waviness of the surface. Both granule segregation and Avicel MCC particle size induced the Ps parameter, which is a measure of the distance between grooves primarily caused by granules of variable sizes. Higher Ps values of the lower surface of the compacts indicate that surface waviness is the dominant factor in determining the Ps parameter. Ps increased with an increase in particle size from Avicel PH-105 to Avicel PH-200. This is probably because larger-size Avicel MCC provides greater spacing between the particles. Because of powder segregation, the lower punch is exposed to a larger portion of the granules that contain the lipid-based formulation. This in turn provides lubrication to the surface of the punch during tablet compaction and ejection. As a consequence, the roughness profile of the lower surface of the compacts given as Ra and Rz was lower than that of the upper surface of the tablets exposed to the less-lubricated upper punch. However, the Avicel MCC particle size was less significant in terms of roughness parameters. This outcome is attributable to the fact that Ra and Rz are measures of the heights and depths of the peaks and valleys formed on the surface of the tablets as a result of powder compaction. Plastic deformation might have diminished the differences between Avicel MCC particles when they were monitored vertically yet maintained their characteristic boundaries detected by the P-profile parameters.

Powdered self-emulsified dosage forms provide an attractive alternative to filled-capsule preparations. The proper excipients selection, however, is crucial when formulating dry adsorbed solid formulations. Greater waviness of the lower surface of the tablets resulted from the segregation of the granules during the die filling. The effect of Avicel MCC particle size was evident on the Ps profile parameter. However, this effect was negligible when evaluated using the R-profile parameter.

### EXAMPLE 3

Aqueous pseudolatex systems are advantageous over organic-based coating systems because aqueous systems are devoid of criteria pollutants such as carbon monoxide, nitrogen oxides, nonmethane volatile organic compounds, and sulfur dioxide. Cellulose acetate butyrate (CAB), which is available from FMC Corporation and Eastman Chemical Company, has been used for organic-based coatings for controlled drug delivery. A pseudolatex was prepared with aqueous based CAB and polyvinyl alcohol (stabilizer) by a polymer emulsification technique. Surface roughness parameters of the pseudolatex CAB coatings on inert Nu-Pareil beads were measured as a function of coating weight gain.

CAB pseudolatex was prepared by the polymer emulsification method. The CAB selected had an acetyl content of 13% w/w and a butyryl content 37% w/w, and had the lowest T_{g} among all available CABs and cellulose acetate polymers. This acetyl and butyryl proportion of CAB imparts a good blend of both hydrophobic and permeability properties to the film. The CAB used had a degree of substitution of 2.9. The Mw was 213,000 and the Mn was 64,500. The polydispersity ratio (Mw/Mn) was 3.3. Accurately weighed 120 g of CAB was dissolved in 480 mL of ethyl acetate and the solution obtained was dispersed in 800 mL of 1% aqueous PVA at 37±1° C., using a homogenizer (model Pro-250; Pro Inc., Monroe, Conn.) at 1000 rpm. The emulsion was passed through a microfluidizer (model M-100Y; Microfluidics Corp., Newton, Mass.) several times and the decreases in globule size were measured after each cycle. The inlet air pressure was maintained at 100 psi. This process was continued until a constant globule size was achieved. The pseudolatex was obtained by stripping the ethyl acetate from this emulsion in a rotary evaporator (model Rotavapor R-114; Brinkmann Instruments Co., Westbury, N.Y.) under reduced pressure. The pseudolatex obtained was suitably diluted and analyzed for polymeric particle size by using a Nicomp Submicron Particle Size Analyzer (model Nicomp 370; Particle Sizing System Inc., Santa Barbara, Calif.).

Approximately 600 g of inert Nu-Pariel beads were used as the initial core to achieve drug loading. The drug-loading suspension consists of verapamil HCL (20% w/w), polydextrose/HPMC (Opadry II) (12% w/w), and talc (2% w/w). A fluidized bed coater (model Strea 1; Nitro Inc., Columbia, Md.) was used for drug loading and controlled-release coating. The following operating parameters were selected: method, bottom spray, spray nozzle diameter, 0.8 mm; atomizing pressure, 0.75 atm; air volume 70 m³/h; and inlet temperature, 40° C. After the drug layering, the beads were dried for 15 min at 45° C. CAB plasticized pseudolatex was used for controlled-release coating. The following operating parameters were selected: method, bottom spray; spray nozzle diameter, 0.8 mm; air volume, 70 m³/h; outlet temperature, 40° C.; atomizing pressure, 0.5 bar; plasticizer concentration, 100% to the solids content of the pseudolatex; and duration of curing, 36 h at 40° C. Beads were sampled out periodically as a function of coating weight gain and the roughness was characterized.

Surface roughness parameters were measured using a Mahr Perthometer concept, a surface roughness-measuring instrument consisting of a motor-driven contact stylus, X/Y-table PZK for mounting the beads, and software for processing the data. The contact stylus scanned over an area of 0.3136 mm² with a tracing length of 0.56 mm to produce 201 profiles. All the roughness parameters were calculated for every profile, and mean and standard deviations for all 201 profiles were collected to represent the complete topography.

Several roughness parameters, such as arithmetic mean roughness (Ra), mean peak to valley height (Rz), geometric average height from a mean line (Rq), maximum profile peak (Rp), and roughness depth (Rt) were calculated.

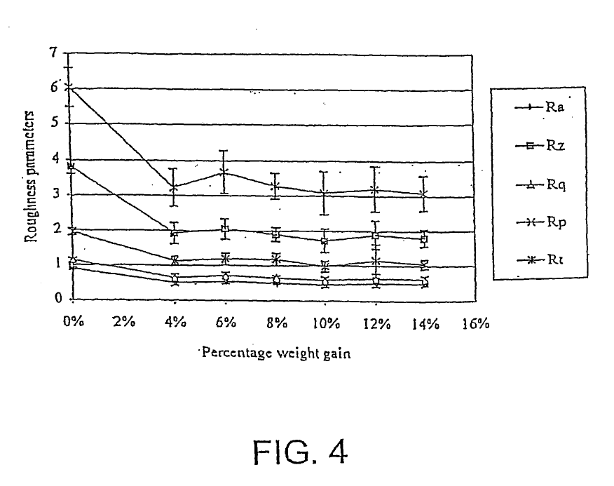

FIG. 4 shows a graph of the roughness parameters plotted as a function of coating weight gain. Initially the surface was rough and, at 4% weight gain, the beads attained a relatively smoother surface. No additional change in roughness was observed as a function of an increase in coating weight gain. The drug-loaded beads appeared to be porous and rough. However, coating reduced the surface roughness and consequently a uniform film was formed.

Although the present invention has been disclosed in terms of a preferred embodiment, it will be understood that numerous additional modifications and variations could be made thereto without departing from the scope of the invention as defined by the following claims:

1. A method for evaluating a dosage form, comprising;
a) determining for a first dosage form at least one surface roughness parameter selected from the group comprising: a mean peak to valley height, a geometric average height from a mean line, a maximum profile peak height, a roughness depth and an arithmetic mean;
b) determining for a second dosage form at least one surface roughness parameter selected from the group comprising a mean peak to valley height, a geometric average height from a mean line, a maximum profile peak height, a roughness depth, and an arithmetic mean roughness;
c) comparing said at least one surface roughness parameter of said first dosage form to a corresponding surface roughness parameter of said second dosage form to obtain a roughness differential; and
d) evaluating a characteristic of said second dosage form based on said roughness differential.

2. The method of claim 1, wherein said dosage form is selected from the group comprising a pharmaceutical dosage form, a herbal dosage form, a nutritional dosage form, and a cosmetic dosage form.

3. The method of claim 1, wherein said step of measuring said second dosage form is performed using a perthometer.

4. The method of claim 1, wherein said dosage form is selected from the group comprising a coated tablet, an uncoated tablet, a cataplasm, a coated bead, an uncoated bead, a granule, a paste, a cream and an ointment.

5. The method of claim 4, wherein said coated tablet has an organic based coating or an aqueous based coating.

6. The method of claim 1, further comprising determining at least two surface roughness parameters selected from the group comprising a mean peak to valley height, a geometric average height from a mean line, a maximum profile peak height, a roughness depth and an arithmetic mean roughness for said second dosage form.

7. The method of claim 1, further comprising determining at least three surface roughness parameters selected from the group comprising a mean peak to valley height, a geometric average height from a mean line, a maximum profile peak height, a roughness depth and an arithmetic mean roughness for said second dosage form.

8. The method of claim 1, further comprising determining at least four surface roughness parameters selected from the group comprising a mean peak to valley height, a geometric average height from a mean line, a maximum profile peak height, a roughness depth and an arithmetic mean roughness for said second dosage form.

9. The method of claim 1, further comprising determining a mean peak to valley height, a geometric average height from a mean line, a maximum profile peak height, a roughness depth and an arithmetic mean roughness for said second dosage form.

10. The method of claim 1, further comprising measuring at least two surface roughness parameters selected from the group comprising a mean peak to valley height, a geometric average height from a mean line, a maximum profile peak height, a roughness depth and an arithmetic mean roughness of said second dosage form.

11. The method of claim 1, further comprising measuring at least three surface roughness parameters selected from the group comprising a mean peak to valley height, a geometric average height from a mean line, a maximum profile peak height, a roughness depth and an arithmetic mean roughness of said second dosage form.

12. The method of claim 1, further comprising measuring at least four surface roughness parameters selected from the group comprising a mean peak to valley height, a geometric average height from a mean line, a maximum profile peak height, a roughness depth and an arithmetic mean roughness of said second dosage form.

13. The method of claim 1, further comprising measuring a mean peak to valley height, a geometric average height from a mean line, a maximum profile peak height, a roughness depth and an arithmetic mean roughness of said second dosage form.

14. The method of claim 1, wherein said characteristic of said second dosage form is selected from the group comprising nature of an ingredient, concentration of an ingredient, process used for preparation, optimum coating end point, grade of an ingredient, coating defect, compression defect, lubricant mixing time, order of mixing an ingredient, and time of mixing an ingredient.

15. The method of claim 1, wherein said at least one surface roughness parameter of said first dosage form is a known surface roughness parameters to provide a standard.

16. The method of claim 15, wherein said at least one surface roughness parameter of said second dosage form is measured.

17. A method for determining a characteristic of a coating for a dosage form comprising,
a) determining at least one roughness parameter for a first dosage form;
b) measuring at least one roughness parameter for a second dosage form;
c) comparing said roughness parameter of said first dosage form to a corresponding roughness parameter of said second dosage form to obtain a roughness differential; and
d) determining said characteristic based on said roughness differential.

18. The method of claim 17, wherein said characteristic is optimum amount of coating or coating defect.

19. The method of claim 17, wherein said roughness parameter for said first dosage form and said second dosage form is selected from the group comprising a mean peak to valley height, a geometric average height from a mean line, a maximum profile peak height, a roughness depth and an arithmetic mean roughness.

20. The method of claim 17, wherein said coating is an organic or aqueous based coating.

21. A method for determining a characteristic of an ingredient in a dosage form comprising,
a) determining at least one surface roughness parameter for a first dosage form;
b) measuring at least one surface roughness parameter for a second dosage form;
c) comparing said roughness parameter of said first dosage form to a corresponding roughness parameter of said second dosage form to obtain a roughness differential; and
d) determining the characteristic of the ingredient based on said roughness differential.

22. The method of claim 21, wherein said ingredient is selected from the group comprising a diluent, a binder, a glidant, a lubricant, and a drug.

23. The method of claim 21, wherein said roughness parameter for said first dosage form and said second dosage form is selected from the group comprising a mean peak to valley height, a geometric average height from a mean line, a maximum profile peak height, a roughness depth and an arithmetic mean roughness.

24. The method of claim 21, wherein said characteristic of an ingredient is selected from the group comprising a nature of the ingredient, a concentration of the ingredient, a grade of the ingredient, an order of mixing of an ingredient, and a time of mixing of an ingredient.

25. A method for determining a process for preparing a dosage form comprising,
a) determining at least one surface roughness parameter for a first dosage form;
b) measuring at least one surface roughness parameter for a second dosage form;
c) comparing said roughness parameter of said first dosage form to a corresponding roughness parameter of said second dosage form to obtain a roughness differential; and
d) determining the process for preparing said dosage form based on said roughness differential.

26. The method of claim 25, wherein said roughness parameter for said first dosage form and said second dosage form is selected from the group comprising a mean peak to valley height, a geometric average height from a mean line, a maximum profile peak height, a roughness depth and an arithmetic mean roughness.

27. The method of claim 25, wherein said process for preparing said dosage form is selected from the group comprising direct compression, wet granulation, and lubricant mixing time.

28. A method for determining a release characteristic of a drug in a dosage form comprising,
a) determining at least one surface roughness parameter for a first dosage form;
b) measuring at least one surface roughness parameter for a second dosage form;
c) comparing said roughness parameter of said first dosage form to a corresponding roughness parameter of said second dosage form to obtain a roughness differential; and
d) determining the release characteristics of a drug based on said roughness differential.

29. The method of claim 28, wherein said roughness parameter for said first dosage form and said second dosage form is selected from the group comprising a mean peak to valley height, a geometric average height from a mean line, a maximum profile peak height, a roughness depth and an arithmetic mean roughness.
